(19) United States
(12) Patent Application Publication
(10) Pub. No.: US 2010/0137715 A1
(43) Pub. Date: Jun. 3, 2010

(54) ULTRASONIC IMAGING APPARATUS AND CONTROL METHOD FOR ULTRASONIC IMAGING APPARATUS

(75) Inventor: Akihiro KAKEE, Nasushiobara-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 12/615,508

(22) Filed: Nov. 10, 2009

(30) Foreign Application Priority Data
Nov. 28, 2008 (JP) 2008-303898

(51) Int. Cl. A61B 8/14

(52) U.S. Cl. 600/443

(57) ABSTRACT

A scanning part ultrasonically scans a cross section of a subject corresponding to a frame period. The filter processor uses time-series received signals that are obtained from the scanning part corresponding to a plurality of frames to attenuate low-frequency components from the received signals of a plurality of locations within the cross section. The amplitude-comparator compares the amplitudes of the received signals with the amplitudes of the signals for which the low-frequency components have been attenuated, and the signals with smaller amplitudes are output. The image-generator generates morphological images of the subject based on the output signals from the amplitude-comparator.

### BACKGROUND OF THE INVENTION

1. Field of the Invention

The present invention relates to: an ultrasonic imaging apparatus that generates ultrasonic cross-sectional images based on at least either phase information or amplitude information included in a received signal obtained by ultrasonically scanning a cross section of a subject; and a control method for an ultrasonic imaging apparatus.

2. Description of the Related Art

Generating a cross-sectional image that shows tissue distribution (i.e., a B mode image) requires two nonlinear processes comprising amplitude wave detection and logarithmic compression of the received signals. Ultrasound radiated within the subject is reflected at the border of acoustic impedance. The reflection intensity is proportional to the difference in the acoustic impedance. When the transmitted ultrasound is considered as a carrier wave, the reflection phenomenon is equivalent to amplitude modulation. Accordingly, by performing wave detection for the amplitudes of the received signals, tissue information can be extracted. As a method of amplitude detection, a nonlinear square wave-detecting method is employed because the received signals are extremely small. Logarithmic compression refers to a process of compressing the dynamic range of a received signal, which can be as much as 2²⁰, for example, into a dynamic range on a relatively small circuit, or substantially into the dynamic range of a monitor.

In a particular ultrasound imaging diagnosis, it is desirable to constrain the parts in which the motion is relatively slow, such as the thoracic wall and the rib bones, and to emphasize only the parts in which the motion is relatively rapid, such as the cardiac wall.

Therefore, as shown in FIG. 1, it has been suggested to place a High Pass Filter (HPF) 014 on the rear stage of both a wave detector 009 and a logarithmic compressor 010, attenuate the relatively low frequencies of fixed echo components (hereinafter referred to as “fixed artifacts”) with relatively low frequencies from the changes over time of the digital signals of each of a plurality of sample points within the cross section, and emphasize the signal echo components having relatively high frequencies.

However, the image data that has been transmitted through the wave detector 009 and the logarithmic compressor 010 has already treated by the nonlinear processing. Therefore, it is impossible to remove the fixed artifacts and to extract only the signal echo components with high accuracy from the image data that has been transmitted through the wave detector 009 and the logarithmic compressor 010. Accordingly, there has been a defect in that the parts to be diagnosed are not clearly extracted from the ultrasonic image.

On the other hand, by implementing a filtering process for the received signal at a filter-processor before it undergoes the nonlinear process, it is possible to sufficiently attenuate specific frequency components. There are techniques (e.g., Japanese Unexamined Patent Application Publication H8-107896) used for the purpose of removing fixed artifacts through this filtering process and displaying only diagnostically beneficial parts clearly. An ultrasonic imaging apparatus that uses this technique also intends to obtain more appropriate filtering characteristics according to the heart time phase. That is, this technique changes the filtering characteristics over time in synchronization with ECG (Electro Cardio Graph) signals. In short, this technique changes the filtering characteristics according to the time-series pattern of the preliminarily set filtering characteristics by utilizing the a priori knowledge that in the time phase when the wall motion is mainly large and the time phase when the wall motion stops, the fluctuation velocity (frequency) of the fixed echo components to be removed and the signal echo components to be extracted are different.

However, techniques such as those described in Japanese Unexamined Patent Application Publication H8-107896 displays a plurality of frames by overlapping them after implementing the filtering process for each frame. Therefore, for signals of a rapidly moving heart valve, for example, a residual image (this may be referred to as a “virtual image of a blurred valve”) is displayed. This results in the generation of an image that contains a blurred virtual image of a valve. When using an image that contains a blurred virtual image of a valve for diagnostic imaging, it may disadvantageously make the evaluation of valve functions difficult. Accordingly, there has been a problem in that it is necessary to switch the filter On and Off depending on the purpose of the diagnosis, complicating the operation; for example, the operator, such as a physician or an operation technician, turns the filter On for evaluations of the cardiac muscles and turns the filter Off for evaluations of valve functions.

### SUMMARY OF THE INVENTION

The present invention aims to provide an ultrasonic imaging apparatus that generates an ultrasound cross-sectional image by removing fixed artifacts and removing blurred virtual images of a valve.

The first embodiment of the present invention is an ultrasonic imaging apparatus comprising: a scanning part that ultrasonically scans a cross section of a subject corresponding to a frame period; a filter-processor that uses time-series received signals that are obtained corresponding to a plurality of frames from the scanning part to attenuate low-frequency components from the received signals of a plurality of locations within the cross section; an amplitude-comparator that compares the amplitudes of said received signals and the amplitudes of signals having attenuated low-frequency components to output signals in which said amplitudes are smaller; and an image-generator that generates morphological images of said subject based on output signals from the amplitude-comparator.

The second embodiment of the present invention is a control method for an ultrasonic imaging apparatus comprising: a scanning step of ultrasonically scanning a cross section of a subject corresponding to a frame period; a filter-processing step of using time-series received signals that are obtained corresponding to a plurality of frames at the scanning step to attenuate low-frequency components from the received signals of a plurality of locations within the cross section; an amplitude-comparing step of comparing the amplitudes of the received signals and the amplitudes of signals for which the low-frequency components have been attenuated to output signals in which the amplitudes are smaller; and an image-generating step of generating morphological images of the subject based on the signals output at the amplitude-comparing step.

The above-mentioned embodiment is configured to generate an ultrasonic cross-sectional image by using received signals and relatively low-amplitude signals among those processed by the filtering process at each point on the scan line. Herein, the blurred virtual image of a valve in the filtering-processed data has larger amplitude than the other parts (high brightness, strong signal). Therefore, in the abovementioned composition, data on the blurred virtual image of a valve in the filtering-processed data is not used and the part is not displayed in the current cross-sectional image data. Accordingly, in the abovementioned composition, it is possible to remove the blurred virtual image of a valve from the ultrasonic cross-sectional image. By doing this, physicians do not have to turn the filtering process On and Off depending on the purpose of a diagnosis. In addition, it allows for easily generating an ultrasonic cross-sectional image in which the fixed artifacts are removed and that is appropriate for the evaluation of valve functions.

### BRIEF DESCRIPTION OF THE DRAWINGS

FIG. 1 is a block diagram of an ultrasonic imaging apparatus according to a conventional example.

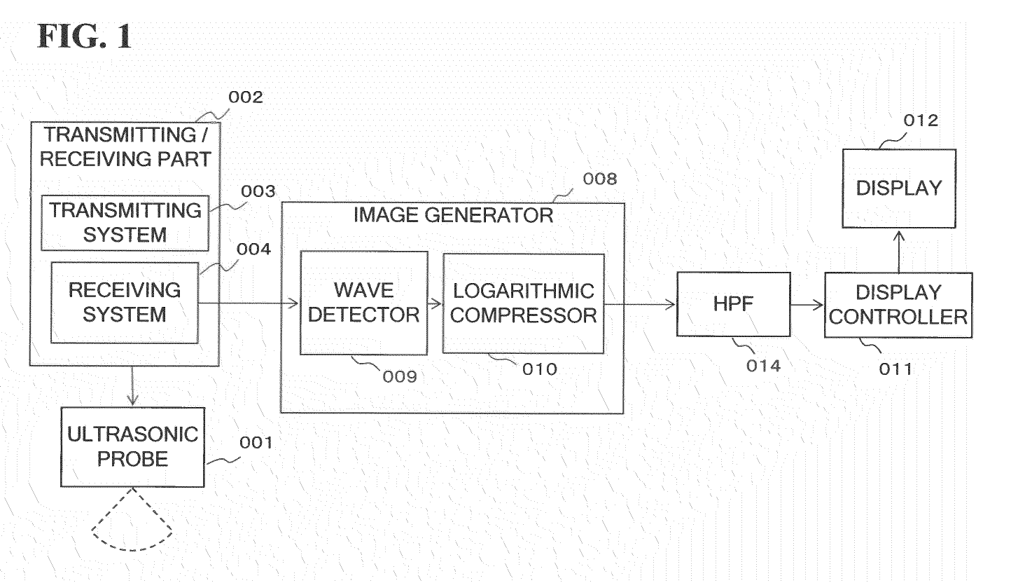

FIG. 2 is a block diagram of an ultrasonic imaging apparatus according to a first embodiment.

FIG. 3a and FIG. 3b are schematic diagrams of filter characteristics.

FIG. 4 is a diagram illustrating an image of a current frame and an image in which low-frequency components have been attenuated.

FIG. 5 is a flow chart of a process for generating an ultrasound cross-sectional image for one frame using an ultrasonic imaging apparatus according to the first embodiment.

FIG. 6 is a block diagram of an ultrasonic imaging apparatus according to a second embodiment.

### DETAILED DESCRIPTION OF THE EMBODIMENTS

### First Embodiment

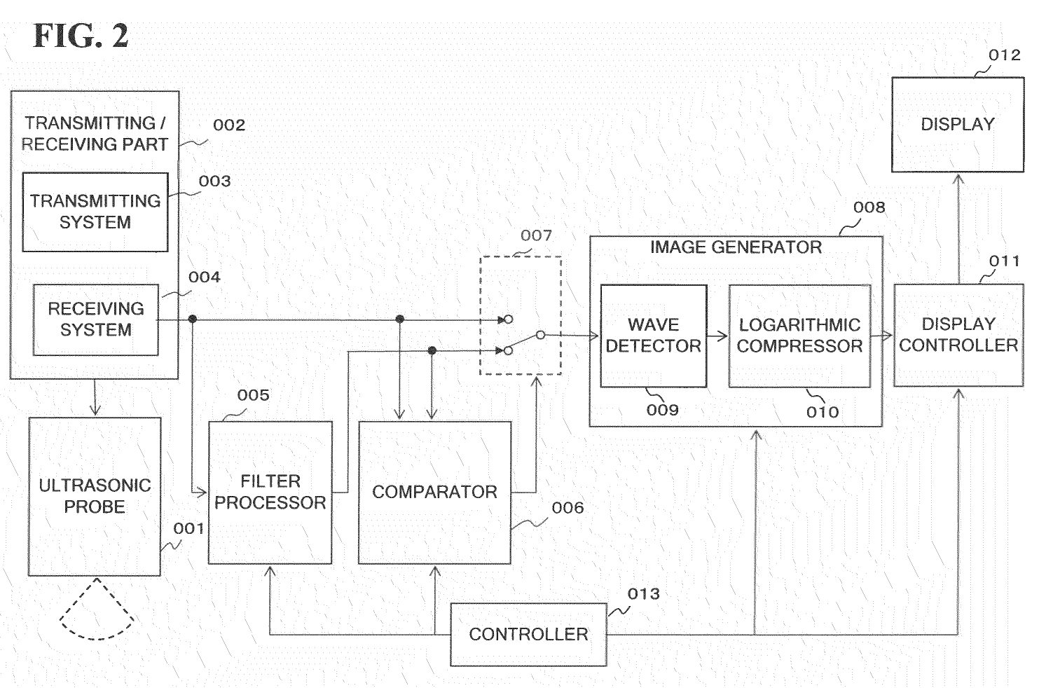

An ultrasonic imaging apparatus according to the first embodiment of the present invention will now be described. FIG. 2 is a block diagram showing the functions of an ultrasonic imaging apparatus according to the present embodiment. Herein, the embodiment is described as one that employs a sector electronic scan mode as the scan mode, but it may be a linear electronic scan mode or a convex scan mode.

On the tip of the ultrasonic probe 001, a plurality of piezoelectric elements that reversibly convert mechanical vibrations and electrical signals are arranged and mounted in one dimension. For each piezoelectric element, a single channel is assigned for scanning a cross section of the subject. This may be a composition in which a plurality of adjacent piezoelectric elements is equivalent to a single channel. The ultrasonic probe 001 is connected to a transmission system 003 of a transmitting/receiving part 002 (described below) during transmission and connected to the receiving system 004 during reception.

When a pulse voltage is applied from the transmission system 003 (described below) to the piezoelectric elements, the ultrasonic probe 001 emits an ultrasonic beam in the direction corresponding to a delay time that is determined by the transmission system 003.

The ultrasound is reflected at the border of acoustic impedance within the subject. This reflected wave is received by the piezoelectric element of the ultrasonic probe 001 to be converted into an electric signal (voltage signal). The ultrasonic probe 001 outputs the generated electric signal to the receiving system 004.

The transmitting/receiving part 002 includes the transmission system 003 and the receiving system 004.

The transmission system 003 has a clock generator, a frequency demultiplier, a transmission delay circuit, and a pulsar, which are not shown. The clock pulse generated by the clock generator is demultiplied by the frequency demultiplier into a 6-KHz rated pulse (trade pulse), for example. The rate pulse output from the frequency demultiplier is allotted into the number of channels by an allotter. The rate pulse output from the allotter can provide different delay times for each channel with the use of the transmission delay circuit. The delay time of each channel is determined by the delay time required for focusing ultrasound to a beam and the delay time according to the direction of transmission of the ultrasonic beam.

By changing the latter delay time, it is possible to scan a fan-like cross section of the subject with the ultrasonic beam. The rate pulse of each channel output from the transmission delay circuit is supplied as a trigger to the pulsars installed for each channel. Each pulsar applies a pulse voltage to the corresponding piezoelectric element of each channel at the timing of receiving the rate pulse.

The receiving system 004 receives an input of electric signals from the piezoelectric element of the ultrasonic probe 001 for each channel. The receiving system 004 has a preamplifier, an analog digital (A/D) converter, a receiving delay circuit, and an adder, which are not shown. The preamplifier, analog digital converter, receiving delay circuit, and adder are all composed as a linear circuit.

The preamplifier amplifies the electrical signal for each channel.

The analog digital converter performs sampling on the amplified electric signal for each channel at a sampling frequency equivalent to, for example, 0.5-mm intervals for each scan line to convert the signals into digital signals for each sampling point. The digital signals can provide different delay times for each channel with the use of the receiving delay circuit.

The delay time of each channel is determined by the delay time required for focusing ultrasound into a beam-like form and the delay time according to the direction of reception of the reflected wave.

Normally, the direction of transmission and the direction of reception are set to be identical. The digital signal of each channel that is output from the receiving delay circuit is added by an adder. Thus, a received signal is obtained in which the reflected components from a particular direction have been emphasized. The received signal contains amplitude information that reflects the differences in acoustic impedance among the tissues and phase information that reflects the motion (moving rate) of the reflector. The received signals output from the receiving system 004 are sent to the wave detector 009 of the image-generator 008.

The combination of the abovementioned ultrasonic probe 001 and the transmitting/receiving part 002, including the transmission system 003 and the receiving system 004, is an example of the “scan part” of the present invention.

The image-generator 008 is equipped with the wave detector 009 and the logarithm compressor 010. The wave detector 009 detects the amplitude of the received signal that has undergone amplitude modulation. By doing this, amplitude information is extracted. For the wave detector 009, because the received signal is extremely small, a nonlinear square wave detection mode is employed. The signal output from the wave detector 009 is sent to the logarithm compressor 010, which acts as a nonlinear circuit. The logarithm compressor 010 compresses the dynamic range of the received signal with the size of, for example, 2²⁰ to the dynamic range on the relatively narrow circuit, or substantially to a relatively narrow dynamic range that a display controller 011 and a display part 012 can handle, to generate image data of a B mode image that reflects the tissue distribution.

The image data generated by the image-generator 008 reflects only amplitude information and does not reflect phase information. At this point, the image data is completely differentiated from the received signal, which includes both types of information. The received signal having amplitude information and phase information is defined as the signal prior to nonlinear processing. The image data is output to the display controller 011.

The display controller 011 causes the display part 012 to display the image data input from the image-generator 008 as a B mode image with a contrasting density.

The filter-processor 005 is provided between the receiving system 004 and the wave detector 009 at the previous stage of the image-generator 008 containing a nonlinear circuit. In addition, the filter-processor 005 uses time-series received signals obtained from the receiving system 004, computes any filtering characteristics regarding each location within the cross section, and is composed as a high-frequency pass digital filter that attenuates particular frequency components.

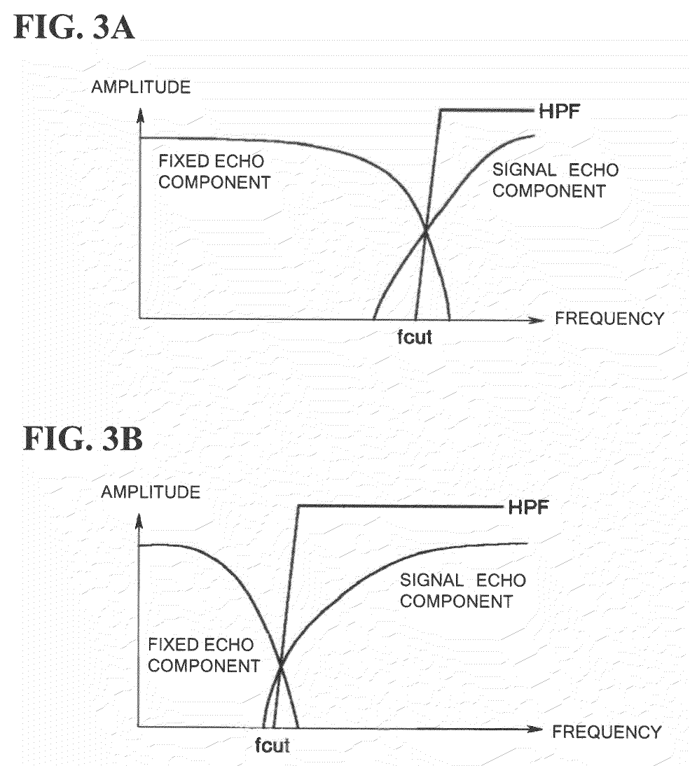

The filter-processor 005 is configured as a high-frequency pass digital filter to attenuate low-frequency components from changes over time (time signals) for each of multiple sample points on the scan line to pass the high-frequency components. This filter removes the fixed artifact by attenuating low-frequency components in the received signal to extract only the signal echo components. The filter-processor 005 has a plurality of filtering characteristics that are defined as cut-off frequencies fcut as shown in FIG. 3a and FIG. 3b. FIG. 3a and FIG. 3b are illustrations of the filtering characteristics. As shown in FIG. 3a and FIG. 3b, because the area for the fixed echo components may be different from that of the signal echo components, in order to remove the fixed echo components in such different areas, a filter with a plurality of filtering characteristics is prepared. The filter-processor 005 is configured operatively by any one of the plurality of filtering characteristics.

The filter-processor 005 has a storage area, such as a memory.

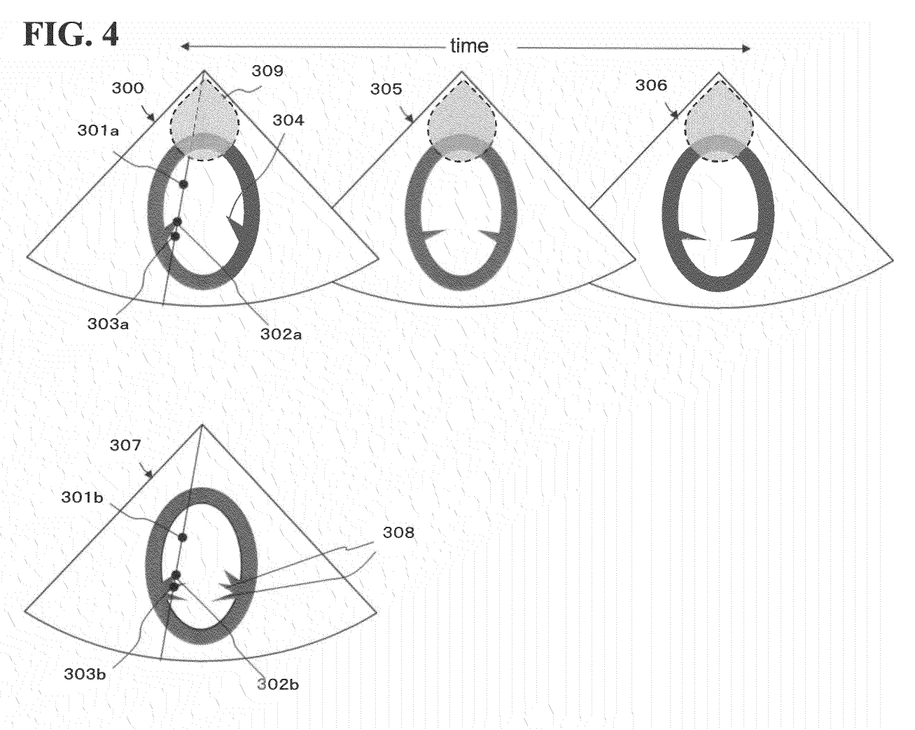

The filter-processor 005 stores the digital signal of each frame for up to 2 frames preceding the current frame. Then, for the current digital signal xi of the same sample point, the digital signal xi-1 of the 1 frame prior to the frame, and the digital signal xi-2 of 2 frames prior to the frame, frequency components lower than the cut-off frequency fcut are attenuated and each signal is added. That is, this filtering process performs a filtering process to the same sample point in each frame. The added result of the filter-processor 005 is output to the image-generator 008 as a digital signal yi of the sample point in which the attenuated low-frequency components are lower than the cut-off frequency according to the filtering characteristics (for details of the filtering process performed by this filter-processor 005, refer to the ultrasonic diagnostic unit described in Japanese Registered Patent No. 3887040 by the applicant of the present application.). FIG. 4 is an explanatory drawing of the current frame image and the image having attenuated low-frequency components.

The image 300 shows a current frame image, the image 305 shows an image of 1 frame prior to the frame, and the image 306 shows an image of 2 frames prior to the frame. Since the image 300, image 305, and image 306 are those obtained before attenuating the low-frequency components, a fixed artifact 309 remains. The image 307 is an image with low-frequency components attenuated. The image 307 is generated by multiplying the attenuated low-frequency components of the image 300, the image 305, and the image 306 with a coefficient and adding. This removes the fixed artifact 309 from the image 307. However, as a result of the abovementioned addition, for such images with low-frequency parts attenuated, such as the image 307, in contrast with the image 300 of the current frame, a blurred virtual image 308 of a valve is displayed (the details will be described below).

The switch 007 performs switching between the receiving system 004 and the wave detector 009 to determine whether or not to bypass the filter-processor 005. That is, the switch 007 switches between the first condition, in which the received signal is supplied from the receiving system 004 via the filter-processor 005 to the wave detector 009, and a second condition, in which the received signal is supplied from the receiving system 004 directly to the wave detector 009.

The comparator 006 is implemented by a CPU. The comparator 006 obtains the digital signal xi at a particular sample point that is output from the receiving system 004 and the digital signal yi at the particular sample point with low-frequency components attenuated that is output from the filtering processor 005. Then, the comparator 006 compares the amplitudes of xi and the amplitudes of yi. Herein, the amplitudes of each digital signal refer to the strength of the signal, which is equivalent to the brightness in cases visualized as an ultrasonic cross-sectional image. If xi is larger than yi (xi>yi), the comparator 006 switches the switch 007 to the first condition to output the digital signal via the filtering processor 005 to the image-generator 008. In addition, if yi is equal to or greater than xi (xi≦yi), the comparator 006 switches the switch 007 to the second condition to directly output the digital signal from the receiving system 004 to the image-generator 008. Herein, as an example of comparing the amplitudes of the digital signals, in particular, the signals output from the receiving system 004 and the filter-processor 005 are I/Q (in-phase/quadrature-phase) signals containing common mode components and orthogonal components, and it is possible to calculate (I²+Q²)^{1/2} for each signal to compare the size of the calculated value.

In the case of the digital signal at the originally identical sample point, for the digital signal passing the filter-processor 005, low-frequency components are attenuated. Therefore, if the compared sample points are in the same condition, the amplitudes becomes smaller than that of the original digital signal (specifically, it can be said that the filtering process in the filter-processor 005 performs addition by multiplying the sample point in each frame by a coefficient and the amplitudes become smaller than that of the original signal in proportion to the amount of attenuation of the low-frequency components). However, because valves move rapidly, valves that should not be present in the current frame are displayed as a blurred virtual part of a valve in the filter-processor 005. Therefore, in the blurred virtual part of a valve, the digital signal via the filter-processor 005 has a larger amplitude than the digital signal directly received from the receiving system 004. Accordingly, by using signals with a smaller amplitude, the blurred virtual part of the valve can be removed.

Herein, referring to FIG. 4, a comparison of the amplitude size at each point will be described in detail. The point 301a and point 301b, point 302a and point 302b, and point 303a and point 303b in the image 300 and the image 307 are respectively the same sample points in the respective images. The point 301a and the point 301b are points in an area where there is nothing other than the heart, the point 302a and the point 302b are points on a heart valve 304, the point 303a is a point in an area where there is nothing other than the heart, and the point 303b is a point on a blurred virtual image 308 of the heart valve. As the point 301a and the point 301b are points in an area where there is nothing other than the heart, low-frequency components are attenuated; therefore, the amplitude of the digital signal at the point 301b becomes smaller. Accordingly, when comparing the point 301a and the point 301b, the comparator 006 switches the switch 007 to the first condition to output digital signals at the point 301b in the image 307 to the image-generator 008. Similarly, at the point 302a and the point 302b, as both are points on the heart valve 304, low-frequency components are attenuated; therefore, the amplitude of digital signals at the point 302b becomes smaller. Accordingly, when comparing the point 302a and the point 302b, the comparator 006 switches the switch 007 to the first condition to output the digital signal at the point 302b in the image 307 to the image-generator 008. In contrast, as the point 303a is a point in an area where there is nothing other than the heart, the amplitude of digital signals is extremely small, while as the point 303b is a point on the blurred virtual image 308 of the heart valve, the amplitude of the digital signals is large. Therefore, when comparing the point 303a and the point 303b, the comparator 006 switches the switch 007 to the second condition to output the digital signal at the point 303a in the image 300 to the image-generator 008. In this way, data in the current frame is used in the blurred virtual parts of the valve, while data for which low-frequency components have been attenuated are used in other parts, which makes it possible to generate an ultrasonic cross-sectional image in which the effects of an artifact as well as the blurred virtual parts of a valve are removed.

Herein, as the switch 007, FIG. 2 illustrates a physical switching apparatus; however, as an example of the actual switching mode, used is a method of having a storage area, such as a memory, storing the digital signals in the current frame from the receiving system 004 and the digital signals of data having attenuated low-frequency components from the filter-processor 005 and, in response to the instructions of the comparator 006, switching the digital signals to be output by outputting either digital signals in the current frame or digital signals of data having attenuated low-frequency components to the image-generator 008.

The controller 013 is implemented by a CPU. The transmission and receiving of data between each functional part is performed via the controller 013. Furthermore, the controller 013 performs timing control of the performance of each functional part and other parts. In addition, the filter-processor 005 is controlled so that the filtering characteristics selected by an operator are applied to the filter-processor 005.

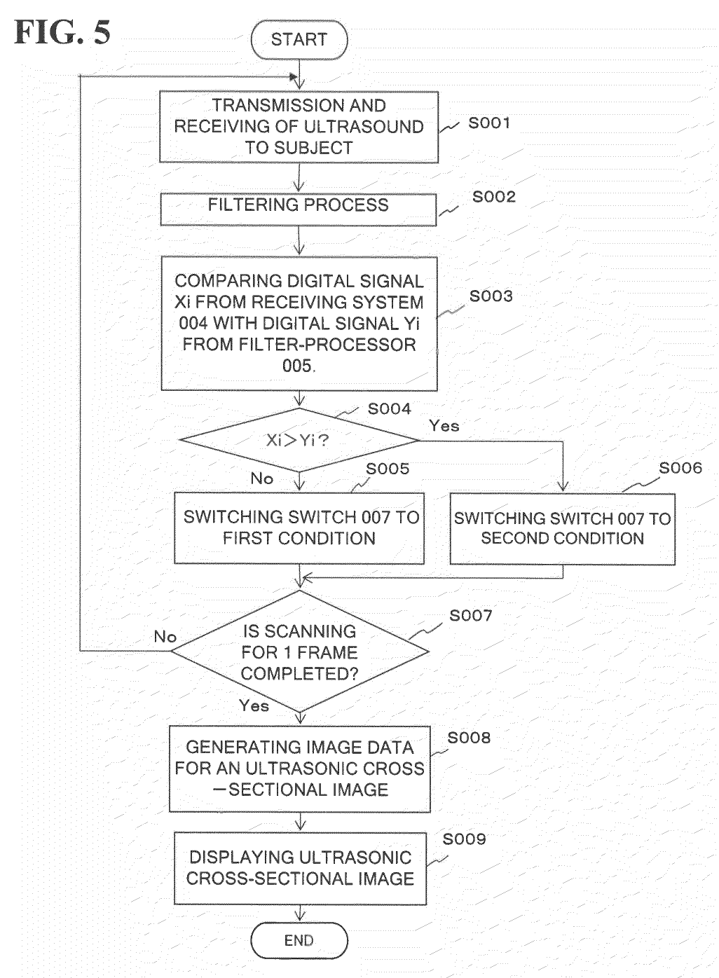

Next, referring to FIG. 5, we will now describe the operations for generating an ultrasonic cross-sectional imaging for 1 frame by an ultrasonic imaging apparatus according to the present embodiment. FIG. 5 is a flow chart showing generation of ultrasonic cross-sectional image for 1 frame by an ultrasonic imaging apparatus according to the present embodiment.

Step S001: Transmission and receiving of the ultrasound is performed by transmitting a pulse signal generated by the transmission system 003 of the transmitting/receiving part 002 to the ultrasonic probe 001, converting it to ultrasound with piezoelectric elements to radiate it to the subject, receiving the ultrasonic echo that is reflected at the border of acoustic impedance by the piezoelectric elements, converting it into an electrical signal, and inputting it into the receiving system 004.

Step S002: The filter-processor 005 performs the filtering process for the digital signal xi output from the receiving system 004.

Step S003: The comparator 006 compares the digital signal xi that is output from the receiving system 004 with the digital signal yi that is output from the filter-processor 005.

Step S004: The comparator 006 determines whether the digital signal xi is larger than the digital signal yi (xi>yi). If xi≦yi, go to Step S005. If xi>yi, go to Step S006.

Step S005: The comparator 006 switches the switch 007 to the first condition to output the digital signal yi that is output from the filter-processor 005 to the image-generator 008.

Step S006: The comparator 006 switches the switch 007 to the second condition to output the digital signal xi that is output from the receiving system 004 to the image-generator 008.

Step S007: The controller 013 determines whether scanning for 1 frame has been completed. If the scanning for 1 frame has been completed, go to Step S008. If the scanning for 1 frame has not yet been completed, return to Step S001.

Step S008: The image-generator 008 generates image data for an ultrasonic cross-sectional image based on the input digital signal xi and the digital signal yi. The image-generator 008 outputs the generated image data to the display controller 011.

Step S009: The display controller 011 causes the display part 012 to display an ultrasonic cross-sectional image based on the image data of the ultrasonic cross-sectional image that is input from the image-generator 008.

As described above, the ultrasonic imaging apparatus according to the present embodiment compares the digital signal at each sample point that is output from the receiving system with the digital signal having attenuated low-frequency components that is output from the filter-processor at the corresponding sample point to use the digital signal with the smaller amplitude for image formation. This allows for forming an image with signals having attenuated low-frequency components in areas other than the area of a rapidly moving valve and removing fixed artifacts. Then, in the area of a rapidly moving valve, an image is formed by the digital signal of the current frame; therefore, it becomes possible to remove the blurred virtual image caused by the blurred valve. Accordingly, this enables physicians to easily refer to images in which fixed artifacts are removed and the blurred virtual image of a valve is removed, which allows for improvements in diagnostic efficiency.

### Second Embodiment

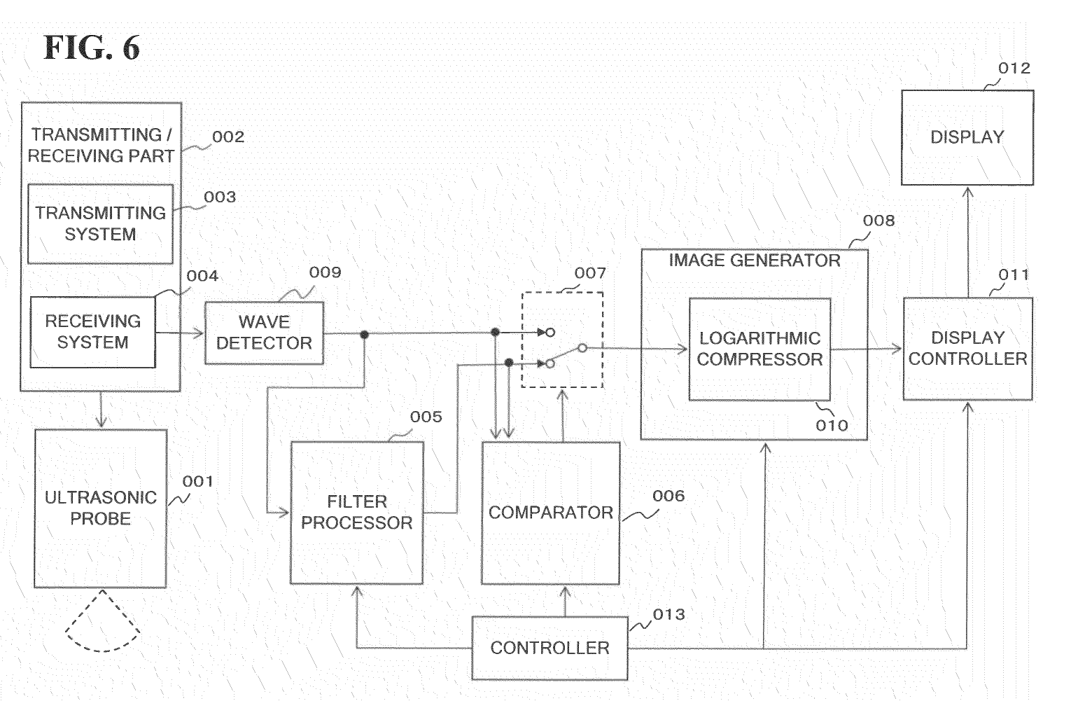

Next, an ultrasonic imaging apparatus according to the second embodiment of the present invention will be described. The ultrasonic imaging apparatus according to the present embodiment is different from the first embodiment in that the filtering process and comparison of digital signals are performed after wave detection. Therefore, the following description will mainly describe signal processing from the receiving system to the switch. In the following description, unless specified otherwise, functioning parts with identical numbers with the first embodiment have the same functions. FIG. 6 is a block diagram showing the functions of the ultrasonic imaging apparatus according to the present embodiment.

The wave detector 009 receives an input of the digital signal xi in the current frame from the receiving system 004. The wave detector 009 detects the wave amplitude of the received signal. This extracts amplitude information. For the wave detector 009, as the received signal is extremely small, a nonlinear square wave detection method is employed. Here, the digital signal xi is an I/Q signal having common-mode components and orthogonal components, and wave detection is performed by obtaining (I²+Q²)^{1/2} for the digital signal xi.

That is, the wave detector 009 performs orthogonal wave detection, wherein wave detection is performed by separating the common-mode component of the I component and the orthogonal component of the Q component.

The filter-processor 005 performs a filtering process for the digital signal xi after wave detection, the digital signal being input from the wave detector 009, to calculate the digital signal yi. This digital signal yi is detected by wave detection.

The comparator 006 obtains the digital signal xi at a particular sample point after wave detection, the digital signal being output from the wave detector 009, and the digital signal yi at the particular sample point for which low-frequency components have been attenuated after wave detection and that is output from the filter-processor 005. Then, the comparator 006 compares the amplitude of xi with the amplitude of yi. When xi is larger than yi (xi>yi), the comparator 006 outputs the digital signal via the filter-processor 005 to the image-generator 008 by switching the switch 007 to the first condition. When yi is equal to or greater than xi (xi≦yi), the comparator 006 directly outputs the digital signal from the receiving system 004 to the image-generator 008 by switching the switch to the second condition. In the present embodiment, because each digital signal has already been converted to (I²+Q²)^{1/2} by the wave detector 009, it is possible to compare the amplitudes as they are.

As described above, the present embodiment involves comparing the amplitudes of digital signals at each sample point after wave detection. This enables the process of amplitude conversion using the comparator to be omitted, thereby allowing for improving the efficiency of forming ultrasonic cross-sectional images.

However, when comparing the first embodiment and the second embodiment, while the first embodiment performs the filtering process for received signals that are received directly from the receiving system, the second embodiment performs the filtering process after wave detection, and therefore, the second embodiment cannot process imaginary numbers and actual numbers separately. Therefore, the accuracy of the filtering process can be improved to a greater degree in the first embodiment. That is, in the first embodiment, it is possible to remove fixed artifacts more accurately and remove blurred virtual parts of a valve.

What is claimed is:

1. An ultrasonic imaging apparatus comprising:
a scanning part configured to ultrasonically scan a cross section of a subject corresponding to a frame period;
a filter-processor configured to use time-series received signals that are obtained corresponding to a plurality of frames from said scanning part to attenuate low-frequency components from the received signals of a plurality of locations within said cross section;
an amplitude-comparator configured to compare the amplitudes of said received signals with the amplitudes of signals for which said low-frequency components have been attenuated to output signals in which said amplitudes are smaller; and
an image-generator configured to generate morphological images of said subject based on output signals from said amplitude-comparator.

2. The ultrasonic imaging apparatus according to claim 1, wherein:
said image-generator further comprises a wave detector configured to perform wave detection for signals that are input from said amplitude-comparator;
said filter-processor and said amplitude-comparator are placed between said scanning part and said wave detector; and
output signals from said filter-processor are input to said wave detector.

3. The ultrasonic imaging apparatus according to claim 1 further comprising a wave detector configured to perform wave detection for said received signals from said scanning part, wherein:
said filter-processor and said amplitude-comparator are placed between said wave detector and said image-generator;
said filter-processor is configured to attenuate low-frequency components of the received signals that are input from said wave detector after wave detection; and
said amplitude-comparator is configured to compare the amplitudes of said received signals after wave detection with the amplitudes of signals for which said low-frequency components have been attenuated to output signals in which said amplitudes are smaller to said image-generator.

4. A control method for an ultrasonic imaging apparatus comprising:
ultrasonically scanning a cross section of a subject corresponding to a frame period;
using time-series received signals that are obtained corresponding to a plurality of frames at said scanning to attenuate low-frequency components from the received signals of a plurality of locations within said cross section;
comparing the amplitudes of said received signals with the amplitudes of signals for which said low-frequency components have been attenuated to output signals in which said amplitudes are smaller; and
generating morphological images of said subject based on the signals output as the result of said comparison.
